(19) United States
(12) Patent Application Publication
(10) Pub. No.: US 2026/0371463 A1
(43) Pub. Date: Nov. 19, 2026

(54) Integrated Lighting System for Ambient Phototherapy

(71) Applicant: Karl S. Jonsson, Rancho Santa Margarita, CA (US)

(72) Inventor: Karl S. Jonsson, Rancho Santa Margarita, CA (US)

(21) Appl. No.: 19/674,918

(22) Filed: May 12, 2026

Related U.S. Application Data
(60) Provisional application No. 63/805,451, filed on May 14, 2025.

(51) Int. Cl. H05B 47/155; A61N 5/06; H05B 45/10; H05B 45/20; H05B 47/13; H05B 47/16; H05B 47/17; H05B 47/175

(52) U.S. Cl. CPC H05B 47/155; A61N 5/0613; H05B 45/10; H05B 45/20; H05B 47/13; H05B 47/16; H05B 47/17; H05B 47/1965; A61N 2005/0626; A61N 2005/0661; A61N 2005/0662

(57) ABSTRACT

A lighting system delivers ambient health therapy as part of everyday illumination. The system combines standard white light with additional sources emitting wavelengths selected for their health benefits, such as UV-B for vitamin D synthesis, near-infrared for tissue recovery, and specific visible colors for mood or circadian regulation. The illumination and therapeutic light sources are independently controlled, enabling pre-defined treatment recipes to cycle through wavelengths over time without interrupting normal lighting. LED groups are individually addressable over a serial bus, and different groups can emit different therapeutic wavelengths. A controller stores and executes recipes, monitors UV exposure for safety, and accepts input from smartphone apps, wall controls, or automation systems. The system also supports a near-infrared mode for invisible camera illumination and a UV disinfection mode that activates when a space is unoccupied.

### CROSS-REFERENCE TO RELATED APPLICATIONS

This application claims the benefit of U.S. Provisional Application 63/805,451 filed May 14, 2025, which is hereby incorporated by reference in its entirety herein for any and all purposes.

### TECHNICAL FIELD

The invention relates to light emitting diode (LED) lighting systems, particularly addressable LED strips, lamps, or luminaires that combine general illumination with integrated health-enhancing wavelengths for ambient phototherapy.

### BACKGROUND

Extensive scientific literature confirms that specific light wavelengths can influence human physiology and well-being. Benefits span across the visible, ultraviolet (UV), and infrared (IR) spectrum, with each wavelength targeting different biological mechanisms.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying drawings, which are incorporated in and constitute part of the specification, illustrate various embodiments. Together with the general description, the drawings serve to explain various principles. In the drawings:

FIG. 1 shows a first example multispectral light emitting diode (LED) apparatus;

FIG. 2 shows a second example implementation of a multispectral LED apparatus;

FIG. 3 shows a third example implementation of a multispectral LED apparatus;

FIG. 4 shows a fourth example implementation of a multispectral LED apparatus;

FIG. 5 shows a fifth example implementation of a multispectral LED apparatus;

FIG. 6 shows a sixth example implementation of a multispectral LED apparatus;

FIG. 7 shows a system block diagram of an example multispectral lighting system including a lighting apparatus, a controller, and a user interface;

FIG. 8 is a flowchart of an example method for providing therapeutic light from an integrated lighting apparatus;

FIG. 9 illustrates an example integrated lighting apparatus operating in a normal mode and in a surveillance illumination mode; and

FIG. 10 is a flowchart of an example method for managing ultraviolet light emission in an integrated lighting apparatus.

### DETAILED DESCRIPTION

In the following detailed description, numerous specific details are set forth by way of examples in order to provide a thorough understanding of the relevant teachings. However, it should be apparent to those skilled in the art that the present teachings may be practiced without such details. In other instances, well known methods, procedures and components have been described at a relatively high-level, without detail, in order to avoid unnecessarily obscuring aspects of the present concepts. A number of descriptive terms and phrases are used in describing the various embodiments of this disclosure. These descriptive terms and phrases are used to convey a generally agreed upon meaning to those skilled in the art unless a different definition is given in this specification. Some descriptive terms and phrases are presented in the following paragraphs for clarity.

Ultraviolet (UV) light refers to electromagnetic radiation having a wavelength between about 100 and about 400 nm. Although not all sources agree on exact definitions, as the terms are used herein, UV-C light refers to electromagnetic radiation having a wavelength between 200 and 280 nanometers (nm), UV-B light refers to electromagnetic radiation having a wavelength between 280 and 315 nm, and UV-A light refers to electromagnetic radiation having a wavelength between 315 and 400 nm. UV light, including UV-A, UV-B, and UV-C light, is invisible to most humans although some people, especially children and young adults, may be able to perceive some UV-A light. While UV-A and UV-B may have some cosmetic or health benefits, UV-C is useful as a disinfectant but has no known health benefits to humans

Visible light refers to electromagnetic radiation having a wavelength between about 400 nm and about 750 nm, depending on the individual viewer. Although not all sources agree on exact wavelengths to represent colors, as the terms are used herein, violet light has a wavelength of below about 450 nm and may overlap with the lower end (i.e. longer wavelengths) of the UV-A band, blue light has a wavelength between about 450 nm and 485 nm, cyan light has a wavelength between about 485 nm and 505 nm, green light has a wavelength between about 505 nm and 565 nm, yellow light has a wavelength between about 565 nm and 590 nm, orange (or amber) light has a wavelength between about 590 nm and 625 nm, and red light has a wavelength above 625 nm.

Infrared (IR) light refers to electromagnetic radiation having a wavelength between about 750 nm and about 1 millimeter (mm). Although not all sources agree on exact definitions, as the terms are used herein, near-infrared (NIR) light has a wavelength below about 3 micrometers (μm), mid-infrared (MIR) light has a wavelength between about 3 μm and 50 μm, and far-infrared (FIR) light has a wavelength above about 50 μm.

Different wavelengths of light are thought to provide different benefits to a human being. Table 1 below lists benefits of different wavelengths of light. The wavelengths are listed in nanometers (nm) and are meant to be interpreted as approximate values, such as +10%.

TABLE 1

| Wavelength | Light | |
|---|---|---|
| (nm) | Type | Benefits |
| 305 | UV-B | Stimulates vitamin D synthesis in the skin, promotes the |
| | | immune system and bone health. |
| 365 | UV-A | Provides cosmetic tanning, supports mood, and may help |
| | | lower blood pressure via nitric oxide release. |
| 405 | Indigo/ | Antimicrobial effects; kills bacteria, supports acne |
| | Violet | treatment and sterilization. |
| 450 | Blue | Regulates circadian rhythm, suppresses melatonin, boosts |
| | | alertness and treats Seasonal Affective Disorder (SAD). |
| 505 | Cyan | Helps alleviate jet lag and fatigue; may aid in melatonin |
| | | phase shifting. |
| 545 | Green | Reduces pain sensitivity; promotes visual comfort and |
| | | reduces eye strain. |
| 575 | Yellow | Enhances mood; warm tone may promote mental well- |
| | | being. |
| 590 | Amber | Preserves night vision; used for low-glare and sleep- |
| | | friendly lighting. |
| 650 | Red | Stimulates healing, reduces inflammation, and enhances |
| | | blood flow. |
| 690 | Deep | Promotes collagen production, wound healing, and tissue |
| | Red | regeneration. |
| 840 | NIR | Deep tissue penetration; improves circulation, reduces |
| | | muscle soreness, may support brain health and |
| | | mitochondrial function. |

Currently, most consumer and clinical devices apply one or two of these wavelengths in single-function, high-intensity units, such as:
Tanning beds for UVA/UVB exposure
Infrared therapy masks and pads
Bright light therapy lamps (10,000 lux) for SAD
Blue light boxes for circadian correction
Deep red/NIR devices for pain and muscle recovery

Existing solutions require user intervention, fixed exposure times, and are often abandoned by users due to inconvenience. They do not allow users to benefit from these health-promoting wavelengths passively as part of their everyday ambient lighting. Most products are limited to circadian-tunable white light (2700K-6500K) or short-use wellness gadgets with limited LED types, and do not allow for integration into smart building lighting systems.

A hybrid lighting system is disclosed herein that combines:
A white or tunable-white light source for ambient illumination;
One or more additional specialty light source, each emitting a targeted health wavelength; and
An addressable control system to deliver specific wavelength “recipes” over time.

While many implementations may utilize light emitting diodes (LEDs) for the light sources, other technologies may be used to generate light inside or outside of the visible spectrum, as is appropriate for a particular application and a current state-of-the-art. The system supports microdosing therapeutic light by embedding low-power specialty LEDs alongside white light sources, distributing them across an addressable pixel architecture (e.g., using SPI-compatible ICs like WS2811, WS2812, UCS8904B).

The system includes one or more groups of light sources (e.g. LEDs). A physical unit may include one or more groups of LEDs. The physical unit may take any form, including an LED strip, a two-dimensional array of pixels with at least some of the pixels being a group of LEDs, a traditional light bulb with an Edison base, a luminaire or lighting fixture for a hard-wired installation, or any other physical form appropriate for a particular application. In some implementations, the different LEDs of a group may be in separate physical devices.

A group (or pixel) includes one or more white or tunable-white LEDs (i.e., illumination LEDs) and at least one LED having a function-specific wavelength (i.e., function-specific LEDs). The function-specific wavelength may be one of the wavelengths listed in Table 1 above or may be any other wavelength of light that is included for a health benefit. For the purposes of this disclosure, including the claims, UV-C light is not considered to be a function-specific wavelength. In some cases, a group of LEDs includes one or more illumination LEDs that emit visible light and at least one function-specific LED emitting a wavelength outside of the visible spectrum, such as an ultraviolet or infrared light. Some implementations may include one or more non-white LEDs emitting visible light in place of, or in addition to the white or tunable-white LEDs, for the illumination LEDs.

A group of LEDs is controlled through two or more control inputs, with at least a first input to control the illumination LEDs and at least a second input to control the function-specific LEDs. A lighting system may include any number of groups of LEDs. The groups of LEDs within a lighting system may be homogenous or heterogeneous and may be controlled by the same set of control inputs or have separate control inputs for different groups of LEDs within the lighting system. In some systems, groups of LEDs may be individually addressable and the LEDs within the addressed group of LEDs may be controlled separately.

The system includes a processor, microcontroller, computer, or external gateway (i.e., a controller) to provide control inputs to the group(s) of LEDs within the system. The control inputs may be modulated power signals that provide power to LEDs or may be signals to a power controller that modulates power to one or more LEDs based on a control input. The controller may map pixels and channels to physical LEDs in the system. It may also store pre-defined “treatment recipes” (e.g., Relax, Stimulate, SAD Therapy, Sunlight, Healing, Plant Growth) that provide a predetermined mix of wavelengths from the various LEDs in the system. Some implementations may support user input via smartphone, wall controls, or automation APIs. It may also control exposure duration and intensity for various wavelengths, such as ultraviolet (UV). This may be done for safety reasons (e.g., to limit UVB output to <1 UV Index, with timed exposure cutoffs), for commercial reasons, and/or user preferences.

Unlike conventional lighting or phototherapy equipment, this system can:
Enable continuous, passive health treatment;
Integrate multiple targeted wavelengths in one fixture;
Uses standard addressable LED ICs in a novel health-focused application; and/or
Provide smart control and safety logic to make therapeutic light part of everyday environments.

Reference now is made in detail to the examples illustrated in the accompanying drawings and discussed below.

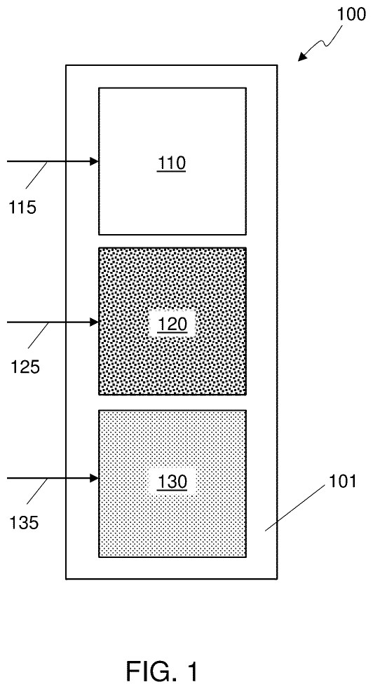

FIG. 1 shows an example multispectral light emitting diode (LED) apparatus 100. The multispectral LED 100 includes three LEDs 110, 120, and 130 mounted on a substrate 101. Any appropriate substrate may be used, but in at least one implementation, the substrate 101 is a 5050 surface-mount device (SMD) package. In other implementations, the substrate may be one or more printed circuit boards that may be rigid or flexible, such as an LED strip substrate. The first LED 110 is an illumination LED that emits white light. The second LED die 120 is a function-specific LED that emits ultraviolet (UV) light, outside of the visible spectrum. The third LED 130 is a function-specific LED that emits deep red light in the visible spectrum or infrared light outside of the visible spectrum. Other implementations may include additional white light LEDs (e.g., a second white LED with a different color temperature than the first white LED) and/or fewer or more function-specific LEDs.

The multispectral LED 100 includes three control inputs, a first control input 115 to control the first LED 110, a second control input 125 to control the second LED 120, and a third control input 135 to control the third LED 130. The inputs 115, 125, 135 may be power inputs or may simply control the other circuitry of the multispectral LED 100 with then provides power to their respective LEDs 110, 120, 130.

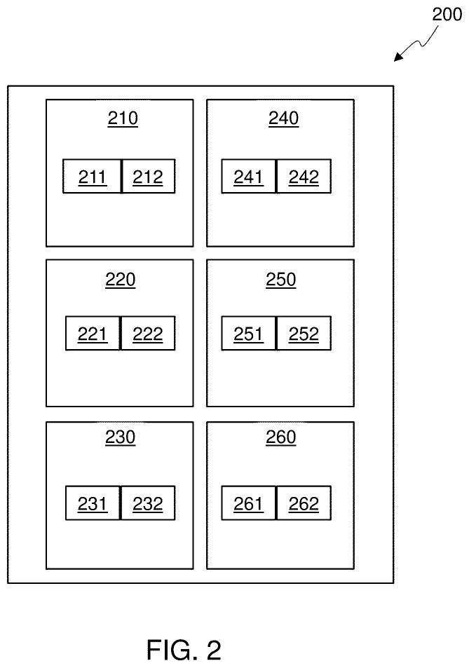

FIG. 2 shows an example alternative implementation of a multispectral LED apparatus 200 that includes 6 pixels, or groups, of LEDs, although the entire apparatus 200 may be looked at as having a single group of 12 LEDs. The first pixel 210 includes a first LED 211 that emits light at 305 nm and a second LED 212 that emits light at 650 nm. The second pixel 220 includes a first LED 221 that emits light at 365 nm and a second LED 222 that emits light at 405 nm. The third pixel 230 includes a first LED 231 that emits light at 450 nm and a second LED 232 that emits light at 505 nm. The fourth pixel 240 includes a first LED 241 that emits light at 545 nm and a second LED 242 that emits light at 575 nm. The fifth pixel 250 includes a first LED 251 that emits light at 590 nm and a second LED 252 that emits light at 690 nm. The sixth pixel 260 includes a first LED 261 that emits light at 840 nm and a second LED 262 that emits white light at color temperature of 3500K. Each of the LEDs in the six pixels can be individually addressed using a serial bus such as Serial Peripheral Interface (SPI), Universal Serial Bus (USB), Inter-Integrated Circuit (I²C), or any other appropriate serial communication protocol. The apparatus 200 may be used to enable custom light treatment recipes to run in the background of normal room lighting.

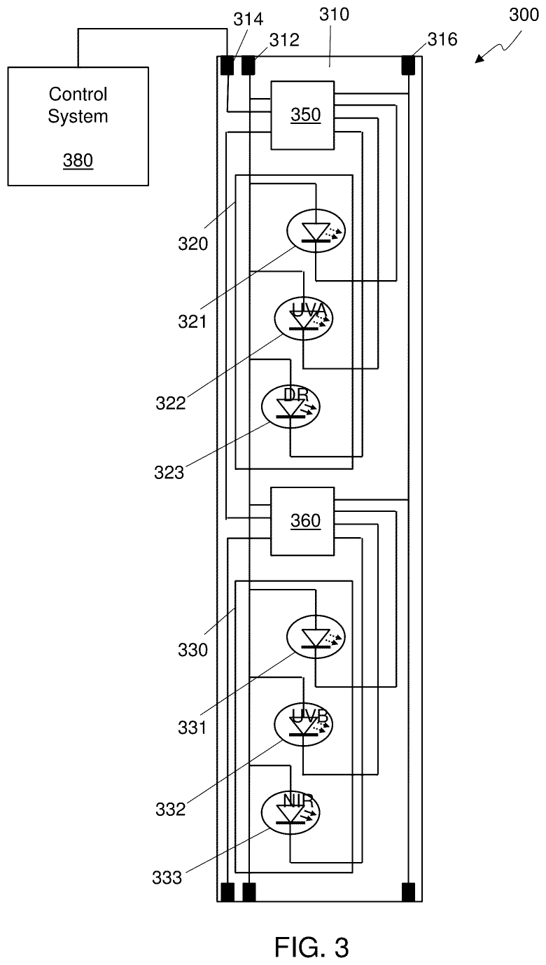

FIG. 3 shows an example of yet another implementation of a multispectral LED apparatus 300 which includes 2 groups of LEDs a first group 320 and a second group 330 mounted on a substrate 310 along with a controller 350. The apparatus 300 may be a section of an LED strip which repeats so that each section has a first group of LEDs 320 coupled to a first controller 350, and a second group of LEDs 330 coupled to a second controller 360. In other implementations a section of the LED strip may include multiple instances of the first group of LEDs 320 and multiple instances of the second group of LEDs 330, coupled to the two respective controllers so that each controller is coupled to multiple groups of LEDs. In some implementations, a single controller may be coupled to both groups of LEDs, with a single controller per section of the LED strip. The apparatus 300 may include a power input 312, a ground input 316, and a control input 314.

The first group of LEDs 320 includes a white phosphor LED 321, a UV-A (e.g., 365 nm) LED 322, and a deep red (e.g., 690 nm) LED. The second group of LEDs 330 includes a white phosphor LED 331, a UV-B (e.g., 305 nm) LED 332, and a NIR (e.g., 840 nm) LED 333. In some implementations, the first group's white LED 321 and the second group's white LED 331 may emit white light at the same color temperature, but in other implementations, they may emit light at different color temperatures.

The first group of LEDs 320 in each section of an LED strip may be considered an odd pixel and the second group of LEDs 330 in each section of the LED strip may be considered an even pixel. Each pixel of the strip may be individually addressed using the control input 314 to send a control signal to the first (i.e., odd) controller 350 which strips out the control information for the first pixel and passes the rest of the control information to the second (i.e., even) controller 360 in a daisy-chained fashion. This allows a control system 380 to command the apparatus 300 to emit white light, deep red light, NIR light, UV-A light, and/or UV-B light, each at a designated intensity under control of the controllers 350, 360. This allows various recipes of light to be produced which can vary over time, under control of the control system 380.

Control system 380 may include dedicated microcontroller or external gateway that:
Maps pixels and channels to physical wavelengths
Stores pre-defined “treatment recipes” (e.g., Relax, Stimulate, SAD Therapy, Sunlight, Healing, Plant Growth)
Supports user input via smartphone, wall controls, or automation APIs
Controls exposure duration and intensity, especially for UV safety (e.g., limits UV-B output to <1 UV Index, with timed exposure cutoffs)

Unlike conventional lighting or phototherapy equipment, this system:
Enables continuous, passive health treatment
Integrates multiple targeted wavelengths in one fixture
Uses standard addressable LED ICs in a novel health-focused application
Provides smart control and safety logic to make therapeutic light part of everyday environments

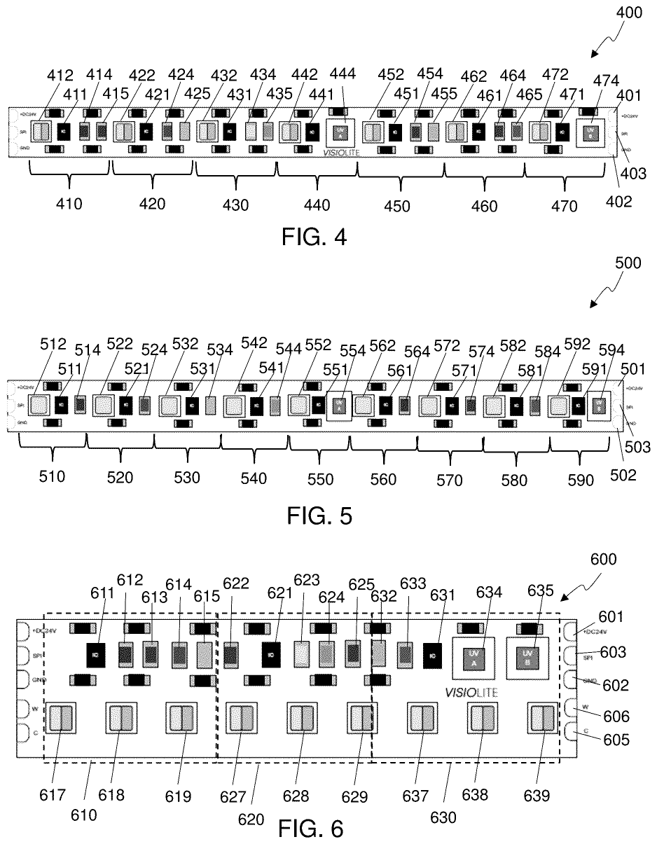

FIG. 4 shows a fourth example implementation of a multispectral LED apparatus 400 which may be a section of an LED strip that repeats the section shown for additional sections. It includes seven groups of LEDs and has a power input 401, a ground input 402 and a serial data input 403. A first group of LEDs 410 is controlled by a first driver integrated circuit (IC) 411 and includes a first tunable white LED 412 having warm white (e.g., 2700K) phosphor LED and a cool white (e.g., 5000K) phosphor LED, a NIR LED 414 and a deep red LED 415. A second group of LEDs 420 is controlled by a second driver IC 421 and includes a second tunable white LED 422, a red LED 424 and an amber LED 425. A third group of LEDs 430 is controlled by a third driver IC 431 and includes a third tunable white LED 432, a yellow LED 434 and a green LED 435. A fourth group of LEDs 440 is controlled by a fourth driver IC 441 and includes a fourth tunable white LED 442, and a UV-A LED 444. A fifth group of LEDs 450 is controlled by a fifth driver IC 451 and includes a fifth tunable white LED 452, a red LED 454 and a cyan LED 455. A sixth group of LEDs 460 is controlled by a sixth driver IC 461 and includes a sixth tunable white LED 462, a blue LED 464 and an indigo LED 465. A seventh group of LEDs 470 is controlled by a seventh driver IC 471 and includes a seventh tunable white LED 472, and a UV-B LED 474. The controllers 411, 421, 431, 441, 451, 461, 471 may be referred to collectively as 4×1 and the tunable white LEDs 412, 422, 432, 442, 452, 462, 472 may be referred to collectively as 4×2.

While the power input 401 and the ground input 402 may connected to conductors that also connect with connectors on the other end, the controllers 4×1 may be coupled in a daisy-chained fashion to the serial data input 403 or may all be individually addressable through a common bus connected to serial data input 403. The controllers 4×1 may each provide 4 individually controllable outputs to drive its associated LEDs, such as using two channels for the two different white LEDs of its associated tunable white LED 4×2 to provide illumination light, and one or two other channels for its other associated LEDs to provide function-specific light. Thus, the multispectral LED apparatus 400 can provide a mix of a color-temperature tuned white light and any of the function-specific wavelengths shown in Table 1 above.

FIG. 5 shows a fifth example implementation of a multispectral LED apparatus 500 which may be a section of an LED strip that repeats the section shown for additional sections. It includes nine groups of LEDs and has a power input 501, a ground input 502 and a serial data input 503 which may be connected similarly to the inputs in apparatus 400. A first group of LEDs 510 is controlled by a first driver integrated circuit (IC) 511 and includes a first white LED 512 and a NIR LED 514. A second group of LEDs 520 is controlled by a second driver IC 521 and includes a second white LED 522 and a red LED 524. A third group of LEDs 530 is controlled by a third driver IC 531 and includes a third white LED 532 and an amber LED 534. A fourth group of LEDs 540 is controlled by a fourth driver IC 541 and includes a fourth white LED 542 and a green LED 544. A fifth group of LEDs 550 is controlled by a fifth driver IC 551 and includes a fifth white LED 552 and a UV-A LED 554. A sixth group of LEDs 560 is controlled by a sixth driver IC 561 and includes a sixth white LED 562 and a deep red LED 564. A seventh group of LEDs 570 is controlled by a seventh driver IC 571 and includes a seventh white LED 572 and a blue LED 574. An eighth group of LEDs 580 is controlled by an eighth driver IC 581 and includes an eighth white LED 582 an indigo LED 584. A ninth group of LEDs 590 is controlled by a ninth driver IC 591 and includes a ninth white LED 492 and a UV-B LED 594.

The controllers 511, 521, 531, 541, 551, 561, 571, 581, 591 (collectively 5×1) may be coupled in a daisy-chained fashion to the serial data input 503 or may all be individually addressable through a common bus connected to serial data input 503. The controllers 5×1 may each provide 2 individually controllable outputs to drive its associated LEDs, using one channel for its associated white LED (5×2) to provide illumination light, and the other channels for its other associated LEDs to provide function-specific light. Thus, the multispectral LED apparatus 500 can provide a mix of a color-temperature tuned white light and any of the function-specific wavelengths shown in Table 1 above.

FIG. 6 shows a sixth example implementation of a multispectral LED apparatus 600 which may be a section of an LED strip that repeats the section shown for additional sections. It includes three groups of LEDs and has a power input 601, a ground input 602 and a serial data input 603 (connected similarly as in apparatus 400 or apparatus 500) as well as a first dedicated drive input 605 for cool white LEDs and a second dedicated drive input 606 for warm white LEDs which may share a common anode (power input 601) or common cathode (ground input 602). A first group of LEDs 610 includes a first tunable white LED 617 having warm white (e.g., 2700K) phosphor LED and a cool white (e.g., 5000K) phosphor LED, a second tunable white LED 618, and a third tunable white LED 619. A second group of LEDs 620 includes a fourth tunable white LED 627, a fifth tunable white LED 628, and a sixth tunable white LED 629. A third group of LEDs 630 includes a seventh tunable white LED 637, a eighth tunable white LED 638, and a ninth tunable white LED 639. The nine tunable white LEDs 617, 618, 619, 627, 628, 629, 637, 638, 639 are each driven directly from the first dedicated drive input 605 for cool white LEDs in the tunable white LEDs and the second dedicated drive input 606 for warm white LEDs in the tunable white LEDs. Other implementations may include different numbers of tunable white LEDs in each group of LEDs and may have some groups of LEDs with no tunable white LEDs.

The first group of LEDs 610 also includes four LEDs that emit function-specific wavelengths that are controlled by a first driver integrated circuit (IC) 611. The function-specific LEDs in the first group of LEDs 610 include a NIR LED 612, a deep red LED 613, a red LED 614 and an amber LED 615. The second group of LEDs 620 also includes four LEDs that emit function-specific wavelengths that are controlled by a second driver IC 621. The function-specific LEDs in the second group of LEDs 620 include a blue LED 622, a yellow LED 623, a green LED 624 and a red LED 625. The third group of LEDs 630 also includes four LEDs that emit function-specific wavelengths that are controlled by a third driver IC 631. The function-specific LEDs in the third group of LEDs 630 include a cyan LED 632, an indigo LED 633, a UV-A LED 634 and a UV-B LED 635.

The controllers 611, 621, 631 (collectively 6×1) may be coupled in a daisy-chained fashion to the serial data input 603 or may all be individually addressable through a common bus connected to serial data input 603. The controllers 6×1 may each provide 4 individually controllable outputs to drive its associated function-specific LEDs while the tunable white LEDs, 617, 618, 619, 627, 628, 629, 637, 638, 639 are each driven directly from the dedicated drive inputs 605, 606. Thus, the multispectral LED apparatus 600 can provide a mix of a color-temperature tuned white light and any of the function-specific wavelengths shown in Table 1 above.

Other implementations may include any number of groups of light sources (e.g., LEDs) each with one or more light sources to emit illumination light in the visible spectrum and one or more light sources to emit function-specific light outside of the visible spectrum. Yet other implementations may include any number of groups of light sources each with one or more light sources to emit illumination light having a broad range of wavelengths in the visible spectrum and one or more light sources to emit function-specific light having a narrow spectrum (e.g., a single color LED).

While the described implementations utilize light emitting diodes (LEDs), other light sources capable of emitting light at targeted wavelengths may be used, including but not limited to laser diodes, organic light emitting diodes (OLEDs), micro-LEDs, quantum dot light emitting diodes (QLEDs), filtered broadband sources such as incandescent, halogen, or fluorescent lamps, plasma light sources, and any other current or future light-generating technology capable of emitting light at a desired wavelength or narrow band of wavelengths with sufficient intensity for the intended illumination or therapeutic application. Use of the word “LED” in the description should not be considered as limiting the example to a light emitting diode for that example.

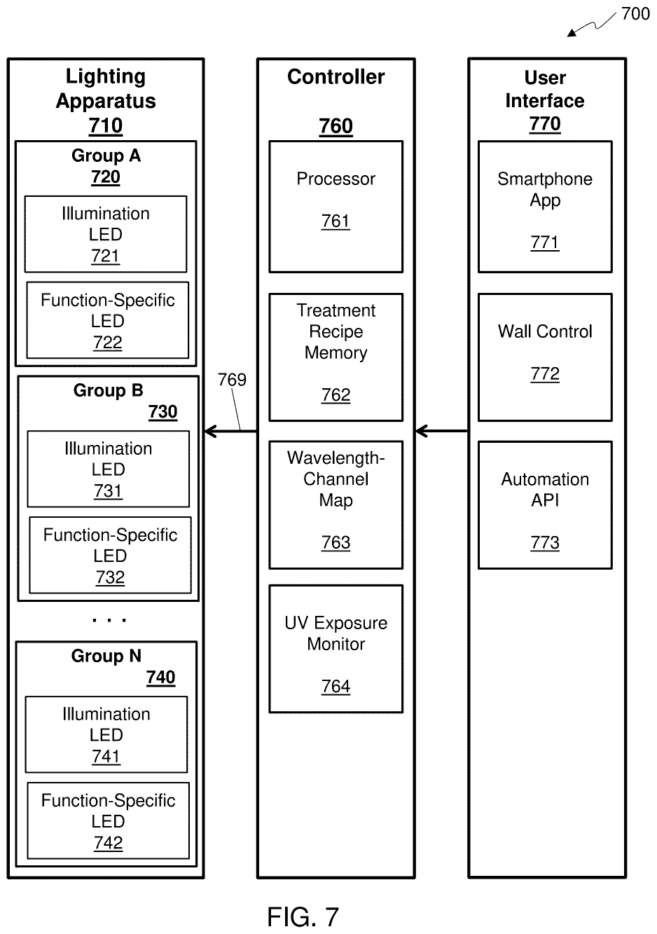

FIG. 7 shows an example system block diagram of a multispectral lighting system 700. The system comprises a lighting apparatus 710 coupled to a controller 760 via a serial control bus 769, and a user interface 770 coupled to the controller 760. The user interface 770 may be coupled to the controller 760 via any usable mechanism, including, but not limited, to the internet, a wired or wireless local area network, a personal area network such as Bluetooth, a wired interface such as USB, or by be integrated into the same device. The lighting apparatus 710 emits general illumination light and function-specific light at wavelengths selected to produce a photobiological effect on a human. The controller 760 manages operation of the lighting apparatus 710 and communicates with the user interface 770 to receive control inputs from users or automated systems.

The lighting apparatus 710 includes a plurality of light source groups, represented in FIG. 7 as group A 720, group B 730, through group N 740, where the ellipsis between group B 730 and group N 740 represents any number of additional groups in a particular implementation. Each group may include an illumination LED that emits general illumination light in the visible spectrum and a function-specific LED that emits function-specific light at a wavelength selected to produce a photobiological effect on a human. Group A 720 includes an illumination LED 721 and a function-specific LED 722. Group B 730 includes an illumination LED 731 and a function-specific LED 732. Group N 740 includes an illumination LED 741 and a function-specific LED 742. In some implementations, the function-specific LEDs 722, 732, 742 in different groups may emit light at different wavelengths from one another, enabling a spatial distribution of therapeutic wavelengths across the lighting apparatus 710. Each group may be individually addressable via the serial control bus 769, enabling the controller 760 to independently control the general illumination light and the function-specific light emitted by each group.

The controller 760 includes a processor 761 and a treatment recipe memory 762. The treatment recipe memory 762 stores a plurality of pre-defined treatment recipes, each specifying at least one target wavelength of function-specific light and a corresponding intensity or duration. Example treatment recipes include Relax, Stimulate, SAD Therapy, Sunlight, Healing, and Plant Growth, among others. The processor 761 selectively executes one or more treatment recipes retrieved from the treatment recipe memory 762, varying the function-specific light emitted by the lighting apparatus 710 over time in accordance with the selected recipe or sequence of recipes.

The controller 760 also includes a wavelength-channel map 763 and may in some cases include a UV exposure monitor 764. The wavelength-channel map 763 maps each target wavelength specified in a treatment recipe to the corresponding group and channel within the lighting apparatus 710, enabling the processor 761 to generate precise control signals for the individual LEDs in the system. The UV exposure monitor 764 tracks cumulative ultraviolet light exposure delivered to occupants of the illuminated space. The controller 760 uses the information from the UV exposure monitor 764 to automatically reduce or terminate UV emission upon reaching at least one of a predetermined time limit, an intensity limit, or a UV Index equivalent threshold, preventing overexposure to ultraviolet light during therapeutic use. Some implementations may also support a timed high-power mode that allows controlled exposure boosts, such as a UV Index equivalent of three to five for a period of fifteen to thirty minutes.

The user interface 770 enables users or automated systems to interact with the controller 760 and may include a smartphone application 771, a wall control 772, or an automation API 773, any or all of which may be used to select a treatment recipe, adjust illumination settings, or configure scheduling and intensity parameters for the general illumination light or the function-specific light. The controller 760 may communicate with the user interface 770 via an open or proprietary API over Bluetooth, Z-Wave, Zigbee, Ethernet, DMX-512A, DALI, RS-485, or any other appropriate wireless or wired interface. Some implementations may support over-the-air (OTA) firmware updates to enable ongoing improvement and expansion of treatment recipes and other features.

In addition to executing treatment recipes for the function-specific light, the controller 760 may also vary the color temperature or intensity of the general illumination light emitted by the illumination LEDs 721, 731, 741 in coordination with execution of a treatment recipe, or independently. Such coordinated illumination control may be used to produce dynamic effects including circadian tuning of color temperature and brightness throughout the day, startup and fade-out sequences, running wave effects or calming pulses for mood or focus, sunrise simulation, and zone-based effects for multi-user environments. These dynamic illumination features may be used alone or in combination with active treatment recipes. Some implementations may be designed to comply with IEC 62471 photobiological safety standards for the wavelengths produced by the function-specific LEDs 722, 732, 742.

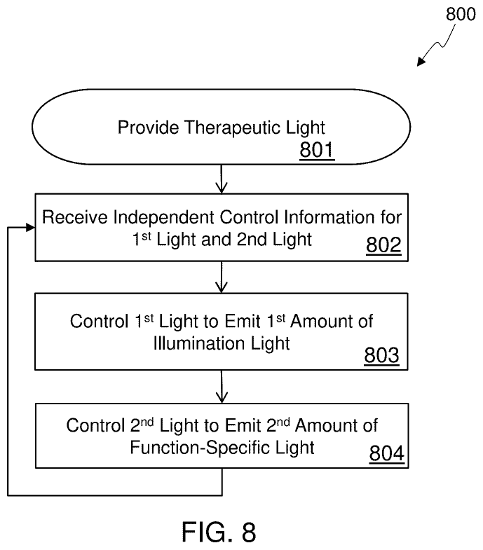

FIG. 8 is a flowchart of an example method 800 for providing therapeutic light 801 from an integrated lighting apparatus having a first light source and a second light source. At step 802, independent control information is received for the first light source and the second light source, specifying separately a first amount for the first light source and a second amount for the second light source. At step 803, the first light source is controlled to emit the first amount of general illumination light in accordance with the independent control information received at step 802.

At step 804, the second light source is controlled to emit the second amount of function-specific light in accordance with the independent control information received at step 802. Because the control information is received and processed independently for the first and second light sources at steps 803 and 804, the amount of general illumination light and the amount of function-specific light may be varied independently of one another. For example, the general illumination light may be dimmed or extinguished without affecting the function-specific light, or the function-specific light may be adjusted to execute a treatment recipe without changing the general illumination. The method 800 may then wait for additional control information at step 802. Traversal through the flowchart 800 from step 802 to step 804 represents the provision of therapeutic light to occupants of the space illuminated by the integrated lighting apparatus.

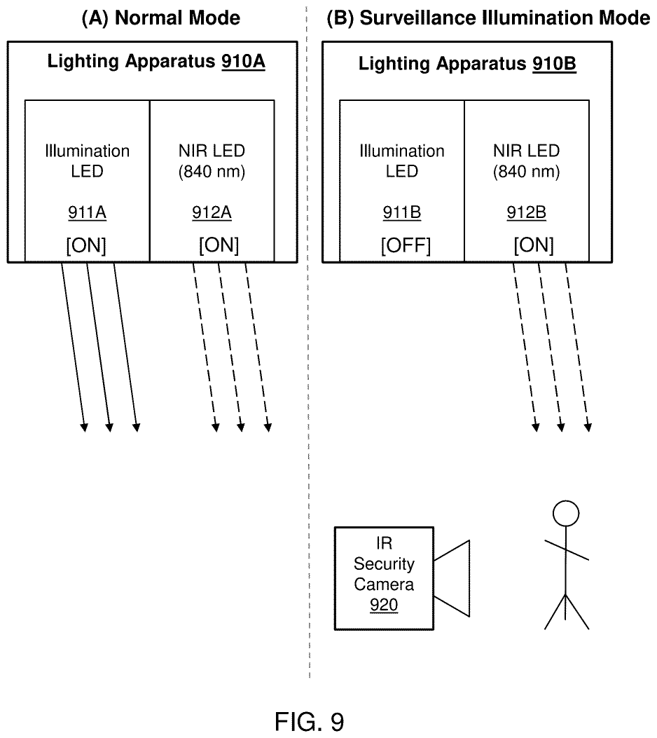

FIG. 9 illustrates an integrated lighting apparatus operating in two different modes. Panel (a) of FIG. 9 shows a lighting apparatus 910A operating in a normal mode in which an illumination LED 911A and a NIR LED 912A are both active. The illumination LED 911A emits general illumination light in the visible spectrum and is shown in an ON state. The NIR LED 912A emits near-infrared light at approximately 840 nm and is also shown in an ON state. In the normal mode, the lighting apparatus 910A simultaneously provides ambient illumination and delivers near-infrared phototherapy to occupants of the space, which may include benefits such as improved circulation, reduced muscle soreness, and support for brain health and mitochondrial function.

Panel (b) of FIG. 9 shows a lighting apparatus 910B operating in a surveillance illumination mode. In this mode, the illumination LED 911B is in an OFF state, while the NIR LED 912B remains in an ON state emitting near-infrared light at approximately 840 nm. Because near-infrared light at approximately 840 nm is invisible to the human eye, the lighting apparatus 910B in the surveillance illumination mode provides invisible illumination compatible with infrared security cameras 920, enabling discreet monitoring of the space without disturbing occupants. The surveillance illumination mode may operate continuously at low intensity or may be triggered by a motion sensor or other event. Because the general illumination light and the NIR light are independently controlled, the NIR LED 912B may remain active while the illumination LED 911B is independently disabled, without requiring any modification to the hardware of the lighting apparatus.

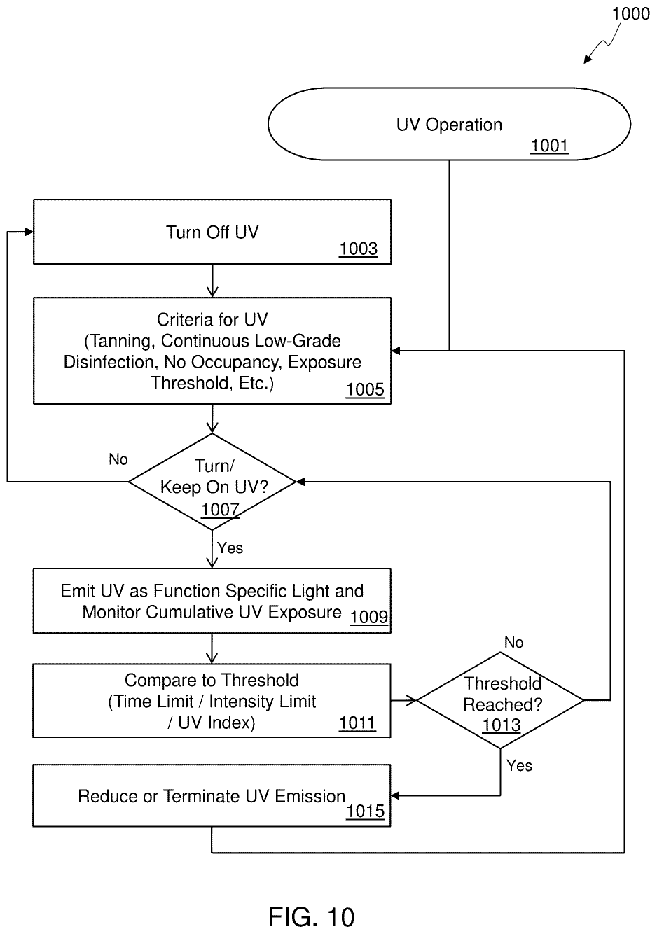

FIG. 10 is a flowchart of an example method 1000 for managing UV light emission in an integrated lighting apparatus. The method 1000 represents a cyclic UV management process applicable to both therapeutic UV use and disinfection. The method 1000 begins at step 1001. At step 1005, criteria for UV operation are assessed. These criteria may include one or more of a treatment recipe requiring UV exposure for a photobiological purpose such as vitamin D synthesis or tanning, a scheduled disinfection operation using UV wavelengths between 315 nm and 450 nm to reduce microbial contamination of surfaces or air within the illuminated space, or an indication of the occupancy status of the illuminated space. The criteria assessed at step 1005 determine the type of UV operation to be performed and the applicable exposure parameters.

At step 1007, it is determined whether or not to turn on (or keep on) the UV light source based on the parameters assessed at step 1005. If it is determined that UV operation should proceed at step 1007, the method 1000 continues to step 1009, at which the second light source is caused to emit UV light as function-specific light and the cumulative UV exposure delivered is monitored. At step 1011, the cumulative UV exposure monitored at step 1009 is compared to an exposure threshold. The exposure threshold may be defined in terms of a time limit, an intensity limit, a UV Index equivalent, or a combination of these, and will vary depending on the type of UV operation selected. At step 1013, if the threshold has not been reached, the method 1000 continues back to step 1007 where other factors may be used to determine whether or not to continue to emit UV light at step 1009, or if the UV should be turned off at step 1003. Once the UV light has been turned off, the method 1000 may wait for other criteria at step 1005 to determine when/whether to turn the UV light back on.

If the threshold for exposure has been reached, the method 1000 goes from step 1013 to step 1015 where the UV emission may be turned off or reduced, depending on the implementation and rules embedded in the system. For example some systems may include two different thresholds where a first threshold is used to reduce the UV light intensity and a second threshold is used to turn off the UV light entirely. Once the UV light has been turned off or reduced at step 1015, the method then loops back to step 1005, where the UV operation type and exposure threshold are reassessed for the next cycle. This cyclic structure enables the lighting apparatus to continuously evaluate whether UV operation is appropriate, to emit UV light when conditions and schedule permit, and to suspend UV emission when the applicable threshold has been reached or when the illuminated space is occupied. In disinfection applications, this allows the lighting apparatus to operate in a disinfection mode during unoccupied periods according to a predetermined schedule and to automatically suspend UV emission when occupancy is detected, independently of the general illumination light, before resuming operation when appropriate conditions return.

Together, the features described above with reference to FIG. 7 through FIG. 10 provide a versatile ambient phototherapy platform that integrates general illumination with independently controlled function-specific light to deliver a broad range of photobiological benefits as part of everyday lighting in residential, commercial, healthcare, and wellness environments, without requiring active user participation. The combination of addressable multi-group architecture, programmable treatment recipes, coordinated illumination control, UV exposure monitoring and safety, disinfection scheduling, and the surveillance illumination mode enables these systems to blend ambient illumination, health support, safety, and automation into a single cost-effective lighting platform.

The systems described herein may serve as a versatile wellness and utility platform, blending ambient lighting, health optimization, safety, and automation into a single cost-effective lighting product. Advantages of such systems may include, but are not limited to, ambient health therapy delivered with zero active user effort, full integration into home, office, or healthcare lighting without disruption to normal illumination, high customization across a broad range of therapeutic wavelengths at low per-wavelength cost using off-the-shelf components, and promotion of occupant well-being, productivity, and convenience. In commercial and institutional settings, such systems may reduce time spent on dedicated therapy sessions compared to active-use phototherapy devices, improve employee wellness and reduce absenteeism, and increase the value and functionality of existing lighting infrastructure.

Multispectral lighting systems as described herein can take any physical form appropriate for a given installation, including but not limited to LED strips for residential and architectural applications, smart bulbs, office and commercial overhead fixtures, and wellness-focused interior environments. In each of these forms, the system integrates white or tunable white light for daily general illumination with therapeutic wavelengths for passive background health support, eliminating the need for separate treatment sessions or dedicated phototherapy equipment.

The addressable multi-wavelength architecture enables standard SPI-addressable driver integrated circuits, such as the WS2812 or UCS8904B, to map individual therapeutic wavelengths to pixel-level channels across an LED strip, lamp, or fixture. Note that “SPI,” as it is used herein, is not limited to a single formal protocol specification, but can refer to any serial interface that supports sending individualized control information to a set of LED drivers, including protocols that allow for specifically addressing individual driver chips, and protocols that use daisy-chained routing to address individual driver chips according to their place in the daisy-chain. This allows a diverse set of therapeutic wavelengths to be spatially distributed across the lighting apparatus and dynamically selected through software-defined treatment recipes. Users may select recipes such as Relax, Energize, Healing, or Sunlight from a smartphone application or automation system, and pre-mapped channel and pixel configurations allow the controller to deliver highly personalized light exposures tailored to individual needs or time-of-day requirements.

The hybrid microdosing approach incorporates safety logic for UV exposure management, including automatic shutoff and UV Index limits, to ensure that therapeutic UV delivery remains within safe parameters during continuous ambient use. Non-therapeutic utility functions, including near-infrared illumination for night vision and security camera support and UV disinfection for surface and air sanitation, further extend the utility of the platform beyond health therapy. The scalable architecture is designed to be deployed in homes, offices, and commercial spaces without disrupting normal lighting aesthetics, and is compatible with building automation systems, smart home platforms, wellness management systems, and ambient intelligence platforms. Together, these characteristics create a new category of ambient phototherapy, transforming the delivery of light-based health support from the domain of specialized clinical or consumer devices into the fabric of everyday lighting infrastructure.

Various examples are described in the following paragraphs:

Example 1: A lighting apparatus comprising: a first light source to emit general illumination light in the visible spectrum; a second light source to emit function-specific light outside of the visible spectrum at a wavelength selected to produce a photobiological effect on a human; and a control input to independently control a first amount of the general illumination light and a second amount of the function-specific light to be emitted by the lighting apparatus.

Example 2: The lighting apparatus of example 1, wherein the first light source and the second light source each comprise an LED.

Example 3: The lighting apparatus of example 1, further comprising a third light source to emit second illumination light in the visible spectrum having a second color temperature; wherein the general illumination light of the first light source is a first illumination light having a first color temperature that is different from the second color temperature; and the control input further controls a third amount of the second illumination light along with the first amount of the first illumination light.

Example 4: The lighting apparatus of example 1, wherein the function-specific light comprises ultraviolet light.

Example 5: The lighting apparatus of example 4, further comprising a controller configured to monitor cumulative ultraviolet light exposure and to automatically reduce or terminate ultraviolet light emission upon reaching at least one of a predetermined time limit, an intensity limit, or a UV Index equivalent threshold.

Example 6: The lighting apparatus of example 1, wherein the function-specific light comprises infrared light.

Example 7: The lighting apparatus of example 6, wherein the function-specific light comprises near-infrared light, and wherein the lighting apparatus is further configured to operate in a surveillance illumination mode in which near-infrared light is emitted without simultaneously emitting general illumination light.

Example 8: The lighting apparatus of example 1, further comprising a plurality of light source groups, each group comprising at least one illumination light source and at least one function-specific light source, wherein each group is individually addressable via a serial control bus to independently control the general illumination light and the function-specific light emitted by that group.

Example 9: The lighting apparatus of example 8, wherein at least two of the plurality of light source groups emit function-specific light at different wavelengths from one another.

Example 10: The lighting apparatus of example 8, wherein the serial control bus operates according to a Serial Peripheral Interface (SPI) protocol, and each group is associated with a driver integrated circuit coupled in a daisy-chained fashion along the serial control bus.

Example 11: The lighting apparatus of example 1, further comprising a controller configured to: store a plurality of pre-defined treatment recipes, a recipe of the plurality of pre-defined treatment recipes specifying at least one target wavelength of function-specific light and a corresponding intensity or duration, and to selectively execute one or more of the treatment recipes to vary the function-specific light emitted by the lighting apparatus over time.

Example 12: The lighting apparatus of example 11, wherein the controller is further configured to receive a user selection of a treatment recipe via at least one of a smartphone application, a wall control, or an automation application programming interface (API).

Example 13: The lighting apparatus of example 11, wherein the controller is further configured to vary the color temperature or intensity of the general illumination light in coordination with execution of a treatment recipe.

Example 14: The lighting apparatus of example 1, wherein the function-specific light has a wavelength greater than 315 nm.

Example 15: The lighting apparatus of example 1, further comprising a controller configured to operate the second light source in a disinfection mode in which function-specific light having a wavelength in the range of 315 nm to 450 nm is emitted according to a predetermined schedule or an indication of occupancy of a space illuminated by the lighting apparatus, independently of the general illumination light, to reduce microbial contamination of surfaces or air within the space illuminated by the lighting apparatus.

Example 16: The lighting apparatus of example 15, wherein the function-specific light emitted in the disinfection mode has a wavelength of approximately 365 nm, approximately 405 nm, or both.

Example 17: A lighting apparatus comprising: a first light source to emit general illumination light having a broad range of wavelengths in the visible spectrum; a second light source to emit function-specific light having a narrow spectrum at a wavelength selected to produce a photobiological effect on a human; and a control input to independently control a first amount of the general illumination light and a second amount of the function-specific light to be emitted by the lighting apparatus.

Example 18: The lighting apparatus of example 17, wherein the narrow spectrum function-specific light is centered at one or more wavelengths selected from approximately 405 nm, 450 nm, 505 nm, 545 nm, 575 nm, and 590 nm.

Example 19: The lighting apparatus of example 17, further comprising a third light source to emit second illumination light in the visible spectrum having a second color temperature; wherein the general illumination light of the first light source is a first illumination light having a first color temperature that is different from the second color temperature; and the control input further controls a third amount of the second illumination light along with the first amount of the first illumination light.

Example 20: The lighting apparatus of example 17, wherein the function-specific light comprises ultraviolet light.

Example 21: The lighting apparatus of example 20, further comprising a controller configured to monitor cumulative ultraviolet light exposure and to automatically reduce or terminate ultraviolet light emission upon reaching at least one of a predetermined time limit, an intensity limit, or a UV Index equivalent threshold.

Example 22: The lighting apparatus of example 17, wherein the function-specific light comprises near-infrared light, and wherein the lighting apparatus is further configured to operate in a surveillance illumination mode in which near-infrared light is emitted without simultaneously emitting general illumination light.

Example 23: The lighting apparatus of example 17, further comprising a plurality of light source groups, each group comprising at least one illumination light source and at least one function-specific light source, wherein each group is individually addressable via a serial control bus to independently control the general illumination light and the function-specific light emitted by that group.

Example 24: The lighting apparatus of example 23, wherein at least two of the plurality of light source groups emit function-specific light at different wavelengths from one another.

Example 25: The lighting apparatus of example 17, further comprising a controller configured to store a plurality of pre-defined treatment recipes, each recipe specifying at least one target wavelength of function-specific light and a corresponding intensity and duration, and to selectively execute one or more of the treatment recipes to vary the function-specific light emitted by the lighting apparatus over time.

Example 26: The lighting apparatus of example 25, wherein the controller is further configured to receive a user selection of a treatment recipe via at least one of a smartphone application, a wall control, or an automation application programming interface (API).

Example 27: The lighting apparatus of example 17, wherein the function-specific light has a wavelength greater than 315 nm.

Example 28: The lighting apparatus of example 17, further comprising a controller configured to operate the second light source in a disinfection mode in which function-specific light having a wavelength in the range of 315 nm to 450 nm is emitted according to a predetermined schedule or an indication of occupancy of a space illuminated by the lighting apparatus, independently of the general illumination light, to reduce microbial contamination of surfaces or air within the space illuminated by the lighting apparatus.

Example 29: The lighting apparatus of example 28, wherein the function-specific light emitted in the disinfection mode has a wavelength of approximately 365 nm, approximately 405 nm, or both.

Example 30: A lighting apparatus comprising: a plurality of light source groups, each group comprising at least one illumination light source to emit general illumination light and at least one function-specific light source to emit function-specific light at a wavelength selected to produce a photobiological effect on a human, wherein each group is individually addressable via a serial control bus to independently control the general illumination light and the function-specific light emitted by that group; and a controller configured to store a plurality of pre-defined treatment recipes, each recipe specifying at least one target wavelength of function-specific light and a corresponding intensity or duration, and to selectively execute one or more of the treatment recipes to vary the function-specific light emitted by the lighting apparatus over time.

Example 31: A method comprising: emitting, by a first light source of an integrated lighting apparatus, general illumination light in the visible spectrum; emitting, by a second light source of the integrated lighting apparatus, function-specific light outside of the visible spectrum at a wavelength selected to produce a photobiological effect on a human; and independently controlling, via a control input of the integrated lighting apparatus, a first amount of the general illumination light and a second amount of the function-specific light emitted.

Example 32: The method of example 31, wherein the function-specific light comprises ultraviolet light configured to provide a phototherapeutic benefit to a user of the integrated lighting apparatus.

Example 33: The method of example 32, further comprising: monitoring cumulative ultraviolet light exposure; and automatically reducing or terminating ultraviolet light emission upon reaching at least one of a predetermined time limit, an intensity limit, or a UV Index equivalent threshold.

Example 34: The method of example 31, wherein the function-specific light comprises near-infrared light, the method further comprising operating in a surveillance illumination mode comprising emitting near-infrared light without simultaneously emitting general illumination light.

Example 35: The method of example 31, further comprising: individually addressing each of a plurality of light source groups of the integrated lighting apparatus via a serial control bus, each group comprising at least one illumination light source and at least one function-specific light source; and independently controlling the general illumination light and the function-specific light emitted by each addressed group.

Example 36: The method of example 35, wherein at least two of the plurality of light source groups emit function-specific light at different wavelengths from one another.

Example 37: The method of example 31, further comprising: storing a plurality of pre-defined treatment recipes, each recipe specifying at least one target wavelength of function-specific light and a corresponding intensity and duration; and selectively executing one or more of the treatment recipes to vary the function-specific light emitted over time.

Example 38: The method of example 37, further comprising receiving a user selection of a treatment recipe via at least one of a smartphone application, a wall control, or an automation application programming interface (API).

Example 39: The method of example 37, further comprising varying the color temperature or intensity of the general illumination light in coordination with execution of a treatment recipe.

Example 40: The method of example 31, wherein the function-specific light has a wavelength greater than 315 nm.

Example 41: The method of example 31, further comprising operating the integrated lighting apparatus in a disinfection mode comprising emitting function-specific light having a wavelength in the range of 315 nm to 450 nm according to a predetermined schedule or an indication of occupancy of a space illuminated by the integrated lighting apparatus, independently of the general illumination light, to reduce microbial contamination of surfaces or air within the space illuminated by the integrated lighting apparatus.

Example 42: The method of example 41, wherein the function-specific light emitted in the disinfection mode has a wavelength of approximately 365 nm, approximately 405 nm, or both.

Example 43: A method comprising: emitting, by a first light source of an integrated lighting apparatus, general illumination light having a broad range of wavelengths in the visible spectrum; emitting, by a second light source of the integrated lighting apparatus, function-specific light having a narrow spectrum at a wavelength selected to produce a photobiological effect on a human; and independently controlling, via a control input of the integrated lighting apparatus, a first amount of the general illumination light and a second amount of the function-specific light emitted.

Example 44: The method of example 43, wherein the narrow spectrum function-specific light is centered at one or more wavelengths selected from approximately 405 nm, 450 nm, 505 nm, 545 nm, 575 nm, and 590 nm.

Example 45: The method of example 43, wherein the function-specific light comprises ultraviolet light configured to provide a phototherapeutic benefit to a user of the integrated lighting apparatus.

Example 46: The method of example 45, further comprising: monitoring cumulative ultraviolet light exposure; and automatically reducing or terminating ultraviolet light emission upon reaching at least one of a predetermined time limit, an intensity limit, or a UV Index equivalent threshold.

Example 47: The method of example 43, wherein the function-specific light comprises near-infrared light, the method further comprising operating in a surveillance illumination mode comprising emitting near-infrared light without simultaneously emitting general illumination light.

Example 48: The method of example 43, further comprising: individually addressing each of a plurality of light source groups of the integrated lighting apparatus via a serial control bus, each group comprising at least one illumination light source and at least one function-specific light source; and independently controlling the general illumination light and the function-specific light emitted by each addressed group.

Example 49: The method of example 48, wherein at least two of the plurality of light source groups emit function-specific light at different wavelengths from one another.

Example 50: The method of example 43, further comprising: storing a plurality of pre-defined treatment recipes, each recipe specifying at least one target wavelength of function-specific light and a corresponding intensity and duration; and selectively executing one or more of the treatment recipes to vary the function-specific light emitted over time.

Example 51: The method of example 50, further comprising varying the color temperature or intensity of the general illumination light in coordination with execution of a treatment recipe.

Example 52: The method of example 50, further comprising receiving a user selection of a treatment recipe via at least one of a smartphone application, a wall control, or an automation application programming interface (API).

Example 53: The method of example 43, wherein the function-specific light has a wavelength greater than 315 nm.

Example 54: The method of example 43, further comprising operating the integrated lighting apparatus in a disinfection mode comprising emitting function-specific light having a wavelength in the range of 315 nm to 450 nm according to a predetermined schedule or an indication of occupancy of a space illuminated by the integrated lighting apparatus, independently of the general illumination light, to reduce microbial contamination of surfaces or air within the space illuminated by the integrated lighting apparatus.

Example 55: The method of example 54, wherein the function-specific light emitted in the disinfection mode has a wavelength of approximately 365 nm, approximately 405 nm, or both.

Unless otherwise indicated, all numbers expressing quantities, properties, measurements, and so forth, used in the specification and claims are to be understood as being modified in all instances by the term “about.” The recitation of numerical ranges by endpoints includes all numbers subsumed within that range, including the endpoints (e.g. 1 to 5 includes 1, 2.78, x, 3.3̅3̅, 4, and 5).

As used in this specification and the appended claims, the singular forms “a”, “an”, and “the” include plural referents unless the content clearly dictates otherwise. Furthermore, as used in this specification and the appended claims, the term “or” is generally employed in its sense including “and/or” unless the content clearly dictates otherwise. As used herein, the term “coupled” includes direct and indirect connections. Moreover, where first and second devices are coupled, intervening devices including active devices may be located there between.

The description of the various embodiments provided above is illustrative in nature and is not intended to limit this disclosure, its application, or uses. Thus, different variations beyond those described herein are intended to be within the scope of embodiments. Such variations are not to be regarded as a departure from the intended scope of this disclosure. As such, the breadth and scope of the present disclosure should not be limited by the above-described exemplary embodiments but should be defined only in accordance with the following claims and equivalents thereof.

We claim as follows:

1. A lighting apparatus comprising:
a first light source to emit general illumination light having a broad range of wavelengths in the visible spectrum;
a second light source to emit function-specific light having a narrow spectrum at a wavelength selected to produce a photobiological effect on a human; and
a control input to independently control a first amount of the general illumination light and a second amount of the function-specific light to be emitted by the lighting apparatus.

2. The lighting apparatus of claim 1, wherein the function-specific light comprises ultraviolet light.

3. The lighting apparatus of claim 2, further comprising a controller configured to monitor cumulative ultraviolet light exposure and to automatically reduce or terminate ultraviolet light emission upon reaching at least one of a predetermined time limit, an intensity limit, or a UV Index equivalent threshold.

4. The lighting apparatus of claim 1, wherein the function-specific light comprises near-infrared light, and wherein the lighting apparatus is further configured to operate in a surveillance illumination mode in which near-infrared light is emitted without simultaneously emitting general illumination light.

5. The lighting apparatus of claim 1, wherein the narrow spectrum function-specific light is centered at one or more wavelengths selected from approximately 405 nm, 450 nm, 505 nm, 545 nm, 575 nm, and 590 nm.

6. The lighting apparatus of claim 1, further comprising a third light source to emit second illumination light in the visible spectrum having a second color temperature;
wherein the general illumination light of the first light source is a first illumination light having a first color temperature that is different from the second color temperature; and
the control input further controls a third amount of the second illumination light along with the first amount of the first illumination light.

7. The lighting apparatus of claim 1, further comprising a controller configured to:
store a plurality of pre-defined treatment recipes, a recipe of the plurality of pre-defined treatment recipes specifying at least one target wavelength of function-specific light and a corresponding intensity or duration, and
selectively execute one or more of the treatment recipes to vary the function-specific light emitted by the lighting apparatus over time.

8. The lighting apparatus of claim 1, further comprising a plurality of light source groups, each group comprising at least one illumination light source and at least one function-specific light source, wherein each group is individually addressable via a serial control bus to independently control the general illumination light and the function-specific light emitted by that group.

9. A lighting apparatus comprising:
a plurality of light source groups, each group comprising at least one illumination light source to emit general illumination light and at least one function-specific light source to emit function-specific light at a wavelength selected to produce a photobiological effect on a human, wherein each group is individually addressable via a serial control bus to independently control the general illumination light and the function-specific light emitted by that group; and
a controller configured to store a plurality of pre-defined treatment recipes, each recipe specifying at least one target wavelength of function-specific light and a corresponding intensity or duration, and to selectively execute one or more of the treatment recipes to vary the function-specific light emitted by the lighting apparatus over time.

10. The lighting apparatus of claim 9, wherein the serial control bus operates according to a Serial Peripheral Interface (SPI) protocol, and each group is associated with a driver integrated circuit coupled in a daisy-chained fashion along the serial control bus.

11. The lighting apparatus of claim 9, wherein at least two of the plurality of light source groups emit function-specific light at different wavelengths from one another.

12. The lighting apparatus of claim 9, wherein the controller is further configured to receive a user selection of a treatment recipe via at least one of a smartphone application, a wall control, or an automation application programming interface (API).

13. A method comprising:
emitting, by a first light source of an integrated lighting apparatus, general illumination light having a broad range of wavelengths in the visible spectrum;
emitting, by a second light source of the integrated lighting apparatus, function-specific light having a narrow spectrum at a wavelength selected to produce a photobiological effect on a human; and
independently controlling, via a control input of the integrated lighting apparatus, a first amount of the general illumination light and a second amount of the function-specific light emitted.

14. The method of claim 13, wherein the function-specific light comprises ultraviolet light configured to provide a phototherapeutic benefit to a user of the integrated lighting apparatus.

15. The method of claim 14, further comprising monitoring cumulative ultraviolet light exposure and automatically reducing or terminating ultraviolet light emission upon reaching at least one of a predetermined time limit, an intensity limit, or a UV Index equivalent threshold.

16. The method of claim 13, wherein the function-specific light comprises near-infrared light, the method further comprising operating in a surveillance illumination mode comprising emitting near-infrared light without simultaneously emitting general illumination light.

17. The method of claim 13, further comprising individually addressing each of a plurality of light source groups of the integrated lighting apparatus via a serial control bus, each group comprising at least one illumination light source and at least one function-specific light source, and independently controlling the general illumination light and the function-specific light emitted by each addressed group.

18. The method of claim 13, further comprising storing a plurality of pre-defined treatment recipes, each recipe specifying at least one target wavelength of function-specific light and a corresponding intensity or duration, and selectively executing one or more of the treatment recipes to vary the function-specific light emitted over time.

19. The method of claim 18, further comprising receiving a user selection of a treatment recipe via at least one of a smartphone application, a wall control, or an automation application programming interface (API).

20. The method of claim 18, further comprising varying the color temperature or intensity of the general illumination light in coordination with execution of a treatment recipe.
